(19) United States
(12) Patent Application Publication
(10) Pub. No.: US 2012/0302809 A1
(43) Pub. Date: Nov. 29, 2012

(54) PRODUCTION OF ALPHA-OLEFINS

(75) Inventors: Joel David Citron, Wilmington, DE (US); Alex Sergey Ionkin, Kennett Square, PA (US)

(73) Assignee: E.I. Dupont De Nemours and Company, Wilmington, DE (US)

(21) Appl. No.: 13/576,732

(22) PCT Filed: Mar. 28, 2011
(86) PCT No.: PCT/US11/30124

§ 371 Date: Aug. 2, 2012

(30) Foreign Application Priority Data
Feb. 3, 2010 (JP) 2010-022209

(51) Int. Cl. C07C 2/26; C07F 15/02; C07D 211/70; C07D 211/78

(52) U.S. Cl. 585/521; 585/527; 546/328; 546/2; 546/329

(57) ABSTRACT

Higher molecular weight linear α-olefins are produced by the oligomerization of ethylene using certain iron complexes of 2,6-diacylpyridinedimimines or 2,6-pyridinedicarboxaldehydedimines as catalysts. These iron complexes are more sterically hindered than those heretofore used. The resulting α-olefins are useful as comonomers in olefin polymerizations.

### CROSS-REFERENCE TO RELATED APPLICATIONS

This application claims the benefit of priority of U.S. Provisional Application Nos. 61/318,556 filed on Mar. 29, 2010; 61/318,570 filed on Mar. 29, 2010; 61/362,563 filed on Jul. 8, 2010; 61/357,362 filed on Jun. 22, 2010; 61/357,368 filed on Jun. 22, 2010; 61/362,563 filed on Jul. 8, 2010 and 61/390,365 filed on Oct. 2, 2010 which are herein incorporated by reference in their entirety.

### TECHNICAL BACKGROUND

Alpha-olefins, or α-olefins, especially those containing 4 to about 20 carbon atoms, are important items of commerce, with about 1.5 million tons reportedly being produced in 1992. The α-olefins are used as intermediates in the manufacture of detergents, as monomers (especially in linear low density polyethylene), and as intermediates for many other types of products.

Most commercially produced α-olefins are made by the oligomerization of ethylene, catalyzed by various types of compounds, see for instance B. Elvers, at al., Ed. Ullmann's Encyclopedia of Industrial Chemistry, Vol. A13, VCH Verlagsgesellschaft mbH, Weinheim, 1989, pp. 243-247 and 275-276, and B. Cornils, at al., Applied Homogeneous Catalysis with Organometallic Compounds, A Comprehensive Handbook, Vol. 1, VCH Verlagsgesellschaft mbH, Weinheim, 1996, pp. 246-256. More recently certain transition metal complexes of diimines of 2,6-pyridinecarboxaldehydes and 2,6-diacylpyridines have been discovered to produce α-olefins by the oligomerization of ethylene, see for instance U.S. Pat. No. 6,103,946, U.S. Pat. No. 6,534,691, U.S. Pat. No. 6,555,723, U.S. Pat. No. 6,683,187 and U.S. Pat. No. 6,710,006, and WO 04/026795, all of which are also incorporated by reference.

As noted above, interest has centered on producing α-olefins having 4 to 20 carbon atoms, more preferably 6 to 12 carbon atoms, as these are considered the most commercially valuable. However, in certain circumstances production of higher α-olefins, say those containing more than 20 carbon atoms, can be advantageous. For example, in the production of polyethylene, the inclusion of long chain branching, which can be derived from higher α-olefins, is believed to improve the processability of the polymer produced, see copending applications 61/318,556 and 61/362,563 which are hereby included by reference. Oligomerization catalysts with higher Schulz-Flory constants are also useful in the production of lubricants and lubricant components, see copending applications 61/357,368 and 61/390,365, which are hereby included by reference. Thus there is a need for a method to produce α-olefins in which a substantial proportion of the product is a “higher” α-olefin.

B. L. Small and M. Brookhart, Macromolecules, 1999, vol. 32, pp. 2120-2130 describe some of the iron complexes used herein in the α-olefin manufacturing process. However they do not suggest that these iron complexes are useful for preparing α-olefins.

### SUMMARY OF THE INVENTION

This invention concerns a process for the manufacture of a series of α-olefins of the formula R⁶⁰CH═CH₂, comprising, contacting ethylene and, optionally, one or more activators and/or cocatalysts, and an iron complex of a ligand of the formula:

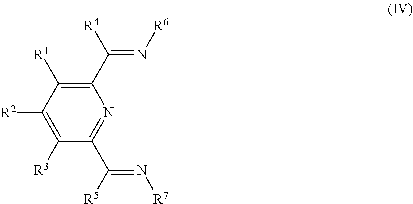

wherein:
R¹, R², and R³ are each independently hydrogen, hydrocarbyl, substituted hydrocarbyl or an inert functional group, provided that any two of R¹, R², and R³ vicinal to one another taken together may form a ring;
R⁴ and R⁵ are each independently hydrogen, hydrocarbyl, substituted hydrocarbyl or an inert functional group provided that R¹ and R⁴ and/or R³ and R⁵ taken together may form a ring;
R⁶⁰ is n-alkyl containing an even number of carbon atoms;
R⁶ is:

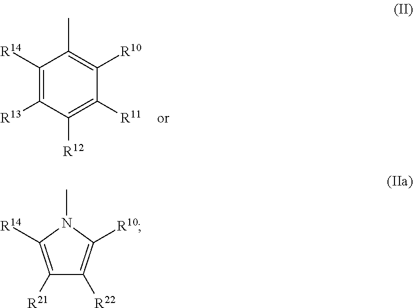

and R⁷ is:

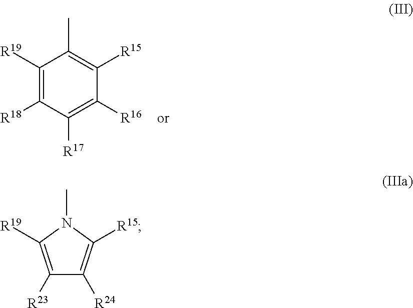

and wherein:
R¹⁰, R¹⁴, and R¹⁵ are each independently hydrocarbyl, substituted hydrocarbyl or a functional group other than fluoro, provided that at least one of R¹⁰, R¹⁴, and R¹⁵ is a secondary carbon group and/or a tertiary carbon group;
R¹¹ to R¹³, R¹⁶ to R¹⁸, and R²¹ to R²⁴ are each independently hydrogen hydrocarbyl, substituted hydrocarbyl or a functional group, and
R¹⁹ is hydrogen or fluoro;

and further provided any two of R¹⁰ through R¹⁹ vicinal to one another may form a ring.

This invention also concerns a compound of the formula

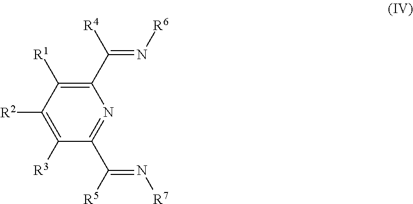

wherein:
R¹, R², and R³ are each independently hydrogen, hydrocarbyl, substituted hydrocarbyl or an inert functional group, provided that any two of R¹, R², and R³ vicinal to one another taken together may form a ring;
R⁴ and R⁶ are each independently hydrogen, hydrocarbyl, substituted hydrocarbyl or an inert functional group provided that R¹ and R⁴ and/or R³ and R⁵ taken together may form a ring;
R⁶⁰ is n-alkyl containing an even number of carbon atoms;
R⁶ is:

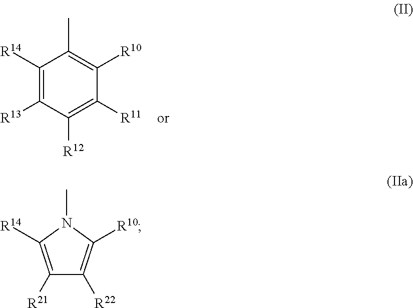

and R⁷ is:

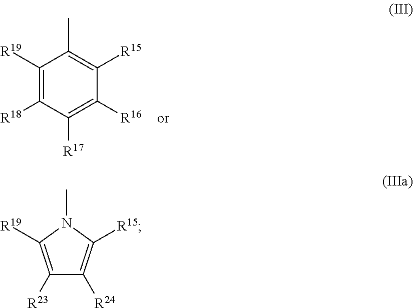

and wherein:
R¹⁰, R¹⁴, and R¹⁵ are each independently hydrocarbyl, substituted hydrocarbyl or a functional group other than fluoro, provided that at least one of R¹⁰, R¹⁴, and R¹⁵ is a secondary carbon group and/or a tertiary carbon group;
R¹¹ to R¹³, R¹⁶ to R¹⁸, and R²¹ to R²⁴ are each independently hydrogen hydrocarbyl, substituted hydrocarbyl or a functional group, and
R¹⁹ is hydrogen or fluoro;

and further provided

any two of R¹⁰ through R¹⁹ vicinal to one another may form a ring;

that R¹⁰, R¹⁴ and R¹⁵ are not all t-butyl;

and that R¹⁰ and R¹⁴ are not t-butyl when R¹⁵ is methyl. Also provided for is an iron complex of (IV).

### DETAILS OF THE INVENTION

In this description certain terms are used and some of them are defined below.

A “hydrocarbyl group” is a univalent group containing only carbon and hydrogen. As examples of hydrocarbyls may be mentioned unsubstituted alkyls, cycloalkyls and aryls. If not otherwise stated, it is preferred that hydrocarbyl groups (and alkyl groups) herein contain 1 to about 30 carbon atoms.

By “substituted hydrocarbyl” herein is meant a hydrocarbyl group that contains one or more substituent groups that are inert under the process conditions to which the compound containing these groups is subjected (e.g., an inert functional group, see below). The substituent groups also do not substantially detrimentally interfere with the polymerization process or operation of the polymerization catalyst system. If not otherwise stated, it is preferred that (substituted) hydrocarbyl groups herein contain 1 to about 30 carbon atoms. Included in the meaning of “substituted” are rings containing one or more heteroatoms, such as nitrogen, oxygen and/or sulfur, where the free valence of the substituted hydrocarbyl may be to the heteroatom. In a substituted hydrocarbyl, all of the hydrogens may be substituted, as in trifluoromethyl.

By “(inert) functional group” herein is meant a group, other than hydrogen, hydrocarbyl or substituted hydrocarbyl, which is inert under the process conditions to which the compound containing the group is subjected. The functional groups also do not substantially deleteriously interfere with any process described herein that the compound in which they are present may take part. Examples of functional groups include halo (fluoro, chloro, bromo and iodo), and ether such as —OR⁵⁰ wherein R⁵⁰ is hydrocarbyl or substituted hydrocarbyl. If the functional group is near a transition metal atom, the functional group alone should not coordinate to the metal atom more strongly than the groups in those compounds that are shown as coordinating to the metal atom, that is they should not displace the desired coordinating group.

By a “cocatalyst” or a “catalyst activator” (or simply “activator”) is meant one or more compounds that react with a transition metal compound to form an activated catalyst species. One such catalyst activator is an “alkylaluminum compound” which herein means a compound in which at least one alkyl group is bound to an aluminum atom. Other groups such as, for example, alkoxide, hydride, an oxygen atom bridging two aluminum atoms, and halogen may also be bound to aluminum atoms in the compound.

By an “α-olefin” is meant a composition predominantly comprising a compound or mixture of compounds of the formula R⁶⁰CH═CH₂ wherein R⁶⁰ is n-alkyl containing an even number of carbon atoms. The product may further contain small amounts (preferably less than 30 weight percent, more preferably less than 10 weight percent, and especially preferably less than 2 weight percent) of other types of compounds such as alkanes, branched alkenes, dienes and/or internal olefins.

By a “series” of α-olefins is meant a series of compounds having the formula R⁶⁰CH═CH₂ wherein at least three compounds, more preferably at least 5 compounds, having different numbers of carbon atoms in R⁶⁰. Preferably in such a series at least some compounds containing R⁶⁰ which contains 2, and 4 and 6 carbon atoms.

By “aryl” is meant a monovalent aromatic group in which the free valence is to the carbon atom of an aromatic ring. An aryl may have one or more aromatic rings, which rings may be fused, connected by single bonds, OF connected to other groups.

By “substituted aryl” is meant a monovalent substituted aromatic group that contains one or more substituent groups that are inert under the process conditions to which the compound containing these groups is subjected (e.g., an inert functional group, see below). The substituent groups also do not substantially detrimentally interfere with the polymerization process or operation of the polymerization catalyst system. If not otherwise stated, it is preferred that (substituted) aryl groups herein contain 1 to about 30 carbon atoms. Included in the meaning of “substituted” are rings containing one or more heteroatoms, such as nitrogen, oxygen and/or sulfur, where the free valence of the substituted hydrocarbyl may be to the heteroatom. In a substituted aryl, all of the hydrogens may be substituted, as in trifluoromethyl. These substituents include (inert) functional groups. Similar to an aryl, a substituted aryl may have one or more aromatic rings, which may be fused, connected by single bonds, or other groups; however, when the substituted aryl has a heteroaromatic ring, the free valence in the substituted aryl group can be to a heteroatom (such as nitrogen) of the heteroaromatic ring instead of a carbon.

The “Schulz-Flory constant” of the mixtures of α-olefins produced is a measure of the molecular weights of the olefins obtained, usually denoted as factor K, from the Schulz-Flory theory (see for instance B. Elvers, et al., Ed. Ullmann's Encyclopedia of Industrial Chemistry, Vol. A13, VCH Verlagsgesellschaft mbH, Weinheim, 1989, pp. 243-247 and 275-276, which is hereby included by reference). This is defined as:

K=(Cₙ₊₂ olefin)/(Cₙ olefin)

wherein (Cₙ olefin) is the number of moles of olefin containing n carbon atoms, and (Cₙ₊₂ olefin) is the number of moles of olefin containing n+2 carbon atoms, or in other words the next higher oligomer of Cₙ olefin. From this can be determined the weight (mass) and/or mole fractions of the various olefins in the resulting oligomeric reaction product mixture.

By a “homopolyethylene” herein is meant a polyethylene made by feeding ethylene as the only polymerizable olefin monomer to the process. Thus a polyethylene made in a process in which ethylene is fed to the process, and some of the ethylene is converted in situ to α-olefins which in turn are copolymerized with ethylene into the polyolefin formed, is a homopolyethylene.

By a secondary carbon group is meant the group:

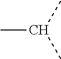

wherein both free bonds represented by the dashed lines are to an atom or atoms other than hydrogen. These atoms or groups may be the same or different. In other words the free valences represented by the dashed lines to may be hydrocarbyl, substituted hydrocarbyl or functional groups. Examples of secondary carbon groups include —CH(CH₃)₂, —CHCl₂, —CH(C₆H₅)₂, cyclohexyl, —CH(CH₃)OCH₃, and —CH═CCH₃.

By a “tertiary carbon group” is meant a group of the formula:

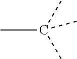

wherein the solid line is the bond to the benzene ring and the three free bonds represented by the dashed lines are to an atom or atoms other than hydrogen. In other words, the bonds represented by the dashed lines are to hydrocarbyl, substituted hydrocarbyl or inert functional groups. Examples of tertiary carbon groups include —C(CH₃)₃, —C(C₆H₅)₃, —CCl₃, —C(CH₃)₂OCH₃, —C≡CH, —C(CH₃)₂CH═CH₂, and 1-adamantyl.

Preferably, in (IV) and its iron complexes, R¹⁰, R¹⁴ and R¹⁵, are each independently alkyl containing 1 to 12 carbon atoms, and/or R¹¹ to R¹³ and R¹⁶ to R¹⁹ are each independently hydrogen or alkyl containing 1 to 12 carbon atoms, and/or R¹, R², and R³ are hydrogen, and/or R⁴ and R⁵ are both methyl or hydrogen, and/or R⁶ is (II), and/or R⁷ is (III). By “at least one of R¹⁰, R¹⁴, and R¹⁵ is a secondary carbon group and/or a tertiary carbon group” is meant that at least one of these three groups is a secondary or tertiary carbon group, but that there may be both tertiary and secondary carbon groups present as R¹⁰, R¹⁴, and R¹⁵. In another preferred form of (IV) and its iron complexes two of R¹⁰, R¹⁴, and R¹⁵ are a secondary carbon group and/or a tertiary carbon group, and it is more preferred that all three of R¹⁰, R¹⁴, and R¹⁵ are a secondary carbon group and/or a tertiary carbon group. In all of these preferred forms, each secondary carbon group and or each tertiary carbon group may be different or the same.

In a preferred for of the iron complex of (IV), only one iron atom and one molecule of (IV) is present.

In an iron complex of (IV), (IV) is usually thought of as a tridentate ligand coordinated to the iron atom through the two imino nitrogen atoms and the nitrogen atom of a pyridine ring. It is generally thought that the more sterically crowded it is about the iron atom the higher the average molecular weight of the α-olefins produced. The present iron complexes have 3 groups in “ortho” positions of the imino groups, and at least one of these is a secondary tertiary carbon group, which is relatively bulky. The molecular weight distribution of the α-olefins produced, as noted above, can be described by the Schulz-Flory constant: the higher the constant, the higher the average molecular weight of the olefins produced. It is believed that under most conditions the present process will have an SFC of about 0.80 to about 0.995.

The synthesis of the ligands (IV) and their iron complexes are well known, see for instance U.S. Pat. No. 6,103,946, G. J. P. Britovsek, et al., cited above, World Patent Application WO 2005/092821, B. L. Small and M. Brookhart, Macromolecules, 1999, vol. 32, pp. 2120-2130, and U.S. Published Application 2006/0178490, all of which are hereby included by reference, and also the Examples herein. These references (except Small) also describe how to carry out α-olefin production with these types of iron complexes.

The steric effect of various groups, such as alkyl groups and other groups, is well know, see for instance R. W. Taft Jr., J. Am. Chem. Soc., vol. 74, pp. 3120-3128 (1952), S. H. Unger, et al., Progress in Physical Organic Chemistry, R. W. Taft, Ed, Vol. 12, John Wiley & Sons, Inc, New York, 1976, pp. 91-101, and Steric Effects in Organic Chemistry, M. S. Newman, Ed., John Wiley & Sons, New York, 1956, pp. 597-603, all of which are hereby included by reference. One need only choose groups according to their steric hindrance based on these and other similar publications in order to produce more or less steric hindrance in ligand and hence in the resulting iron complex.

While steric hindrance about the iron atom is usually the dominant item controlling the SFC, process conditions may have a lesser effect. Higher process temperatures generally give lower SFCs, while higher ethylene pressures (concentrations) generally give higher SFCs, all other conditions being equal.

Table 1 shows the relationship between SFCs and the amounts of α-olefins produced in certain ranges of carbon atom content.

TABLE 1

| SF Constant | 0.75 | 0.85 | 0.95 | 0.98 | 0.99 | 0.995 |
|---|---|---|---|---|---|---|
| Mole percent | | | | | | |
| C50-C100 | 0.13 | 2.35 | 22.65 | 25.84 | 19.88 | 15.27 |
| C50-C200 | 0.13 | 2.38 | 30.13 | 49.63 | 46.17 | 39.59 |
| C50-C300 | 0.13 | 2.38 | 30.70 | 58.29 | 62.08 | 58.53 |
| C50-C400 | 0.13 | 2.38 | 30.70 | 61.44 | 71.70 | 73.26 |
| C50-C500 | 0.13 | 2.38 | 30.70 | 62.59 | 77.52 | 84.87 |
| C100-C200 | 0.00 | 0.04 | 7.91 | 24.55 | 26.91 | 24.87 |
| Weight Percent | | | | | | |
| C4-C50 | 99.56 | 92.95 | 40.48 | 10.94 | 4.34 | 2.36 |
| C4-C100 | 100.00 | 99.79 | 74.91 | 29.59 | 13.63 | 8.03 |

These calculations are fairly exact, using the equation given for the SFC above and other standard stoichiometric calculations. The calculations for SFCs of 0.75 to 0.85 were made out to olefins containing 200 carbon atoms, for an SFC 0.95 olefins out to 300 carbons were calculated, and for SFCs of more than 0.95 calculations were made out to 500 carbon olefins. As can be seen for a SFC of 0.65, little or no olefin containing 50 carbon atoms or more is produced. At a SFC of about 0.75 significant amounts of 050 or higher olefins are produced, and this increases as the SFC increases. As the SFC is raised proportionately lesser and lesser amounts of lower α-olefins are produced (under otherwise the same process conditions), and the amount of higher α-olefins increases.

The present process is useful in making a series of α-olefins in which the relatively high molecular weight olefins predominate. This is particularly useful in making good processing polyethylenes. As mentioned above, for most commercial purposes higher molecular weight α-olefins, those having more than about 14 to 20 carbon atoms are not very useful, and oligomerization catalysts that produce relatively large quantities of these higher olefins (La, have a high SFC) often have to be converted to other products making them considerably less valuable. Therefore commercial processes use oligomerization catalysts that have relatively low SFCs, say about 0.5 to about 0.65, see for instance U.S. Pat. Nos. 5,523,508, 6,501,000, 6,683,187, and 7,053,020, all of which are hereby included by reference.

The present process may be carried out in the presence of another catalyst that copolymerizes ethylene and α-olefins so as to form a branched polyethylene, a homopolyethylene. This process may be carried out as any polymerization process to make polyethylenes, such as gas phase (usually with a supported catalyst) or liquid phase, the latter being possibly slurry or solution process.

Alternatively the present process may be carried out, usually in the liquid phase, in the presence of a solvent to simply produce the desired α-olefins. After optionally removing the solvent, these α-olefins may be added to an olefin polymerization as comonomers. Alternatively the series of α-olefins may be “fractionated” into individual compounds as by distillation, or partially fractionated into portions, each portion containing a range of α-olefins of different molecular weights. A combination in which individual compounds and portions containing molecular weight ranges may also be done. Lower molecular weight α-olefins may be purified by distillation, but the higher molecular weight compounds may be difficult to separate completely. Portions containing higher molecular weight ranges may be produced by fractional crystallization and/or using differential solubility. One or more of these isolated fractions may then be used in an olefin polymerization as comonomer.

The process for making α-olefins using iron complexes of (IV) as described herein may be carried out in the same manner as those processes using similar catalysts but which results in lower SFCs, see for instance U.S. 6,103,946, U.S. Pat. No. 6,534,691, U.S. Pat. No. 6,555,723, U.S. Pat. No. 6,683,187 and U.S. Pat. No. 6,710,006, and WO 04/026795.

In order to measure the Schulz-Flory constant of the present catalyst, the process is carried out. For a catalyst of the present process with a relatively low SFC, say 0.80 to about 0.90, the resulting mixture of α-olefins is analyzed to determine their molecular ratios, and this is most conveniently done by standard gas chromatography using appropriate standards for calibration. Preferably the ratios (as defined by the equation for “K”, above) between olefins from C₆ to C₃₀ (if possible) are each measured and then averaged to obtain the Schulz-Flory constant. If the ratios of higher olefins, such as C₂₀ to C₃₀ are too small to measure accurately, they may be omitted from the calculation of the constant. For catalysts with higher SFCs, say >0.90, it may not be possible to accurately measure the constant from just the olefins up to about C₃₀ since the concentration change from olefin to olefin is relatively small and a broader range may be needed to accurately measure the SFC, i.e., higher olefins need to be measured. Such higher olefins are not very volatile and it may be advantageous to use liquid chromatography (possibly combined with mass spectroscopy to measure what is the particular olefin being eluted), again using appropriate standards for calibration.

In the Examples THF is tetrahydrofuran.

### Example 1

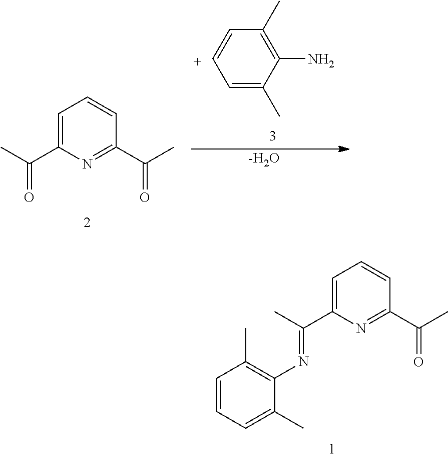

### 1-{6-[1-(2,6-Dimethyl-phenylimino)-ethyl]-pyridin-2-yl}-ethanone (1)

1-(6-Acetyl-pyridin-2-yl)-ethanone 2 (22.2 g, 0.0136 mole), 15.0 g (0.124 mol) of 2,6-dimethyl-phenylamine 3, 300 ml of n-propanol, and a few crystals of p-toluenesulfonic acid were stirred at room temperature for 36 h in 500 ml flask under a flow of nitrogen. The resultant yellow precipitate was filtered and washed by 20 ml of methanol. It was then dried at 1-mm vacuum overnight. The yield of 1-{6-[1-(2,6-dimethyl-phenylimino)-ethyl]-pyridin-2-yl}-ethanone 1 was 12.86 g (39%) as a yellow solid. ¹H NMR (500 MHz, CD₂Cl₂, TMS): δ 2.00 (s, 6H, Me), 2.20 (s, 3H, Me), 2.70 (s, 3H, Me), 6.90 (t, ³J_{HH}=8.1 Hz, 1H, Arom-H), 7.10 (d, ³J_{HH}=8.1 Hz, 2H, Arom-H), 7.95 (t, ³J_{HH}=8.0 Hz, 1H, Pyr-H), 8.10 (d, ³J_{HH}=8.0 Hz, 1H, Py-H), 8.55 (d, ³J_{HH}=8.0 Hz, 1H, Py-H), ¹³C NMR (500 MHz, CD₂Cl₂, TMS (selected signals)): δ 167.1 (C═N), 200.1 (C═O). Anal. Calculated for C₁₇H₁₈N₂O (Mol. Wt.: 266.34): C, 76.66; H, 6.81; N, 10.52. Found: C, 76.69; H, 6.84; N, 10.57.

### Example 2

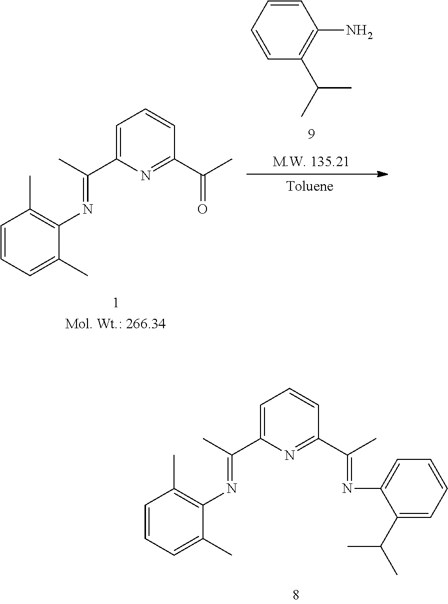

### (2,6-Dimethyl-phenyl)-(1-{6-[1-(2-isopropyl-phenylimino)-ethyl]-pyridin-2-yl}ethylidene)-amine (8)

1-{6-[1-(2,6-Dimethyl-phenylimino)-ethyl]-pyridin-2-yl}-ethanone 1 (5.0 g, 0.0188 mol), 3.30 g (0.0244 mol) of 2-isopropyl-phenylamine 9, 100 g of fresh molecular sieves, and 100 ml of toluene were kept at 100° C. for 3 days under a flow of nitrogen. The solvent was removed in a rotary evaporator and the residue was recrystallized from 20 ml of ethanol. The yield of (2,6-dimethyl-phenyl)-(1-{6-[1-(2-isopropyl-phenylimino)-ethyl]-pyridin-2-yl}-ethylidene)-amine 8 was 4.90 g (68%) as a yellow solid.

### Example 3

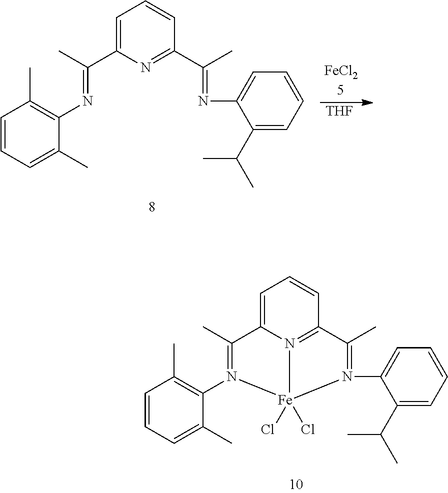

### (2,6-Dimethyl-phenyl)-(1-{6-[1-(2-isopropyl-phenylimino)-ethyl]-pyridin-2-yl}ethylidene)-amine iron (II) chloride (10)

(2,6-Dimethyl-phenyl)-(1-{6-[1-(2-isopropyl-phenylimino)-ethyl]-pyridin-2-yl}-ethylidene)-amine 8 (1.0 g, 0.0026 mol) was added in one portion to the suspension of 0.31 g (0.0025 mol) of iron (II) chloride in 50 ml of THF at ambient temperature in nitrogen glove box. The reaction mixture was stirred for 12 h. The resultant blue solid was filtered and washed by 50 ml of pentanes three times and dried under 1-mm vacuum overnight. The yield of (2,6-dimethyl-phenyl)-(1-{6-[1-(2-isopropyl-phenylimino)-ethyl]pyridin-2-yl}-ethylidene)-amine iron (II) chloride 10 was 1.06 g (85%).

### Example 4

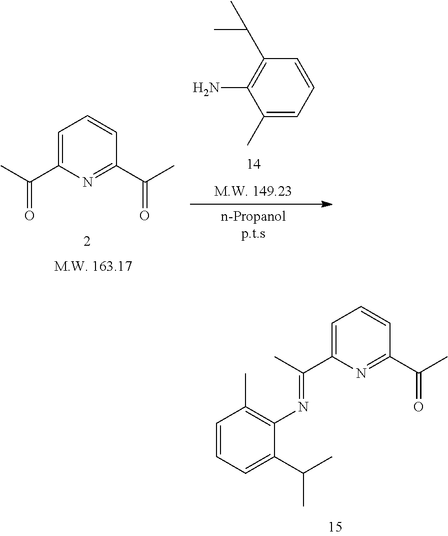

### 1-{6-[1-(2-Isopropyl-6-methyl-phenylimino)-ethyl]-pyridin-2-yl}-ethanone (15)

1-(6-Acetyl-pyridin-2-yl)-ethanone 2 (35.54 g, 0.22 mol), 25.0 g (0.168 mol) of 2-Isopropyl-6-methyl-phenylamine 14, 350 ml of n-propanol, and a few crystals of p-toluenesulfonic acid were stirred at room temperature for 36 h in a 500 ml flask under a flow of the nitrogen. The resultant yellow precipitate was filtered and washed by 20 ml of methanol. It was then dried at 1-mm vacuum overnight. The yield of 1-{6-[1-(2-Isopropyl-6-methyl-phenylimino)-ethyl]-pyridin-2-yl}-ethanone 15 was 13.35 g (27%) as a yellow solid.

### Example 5

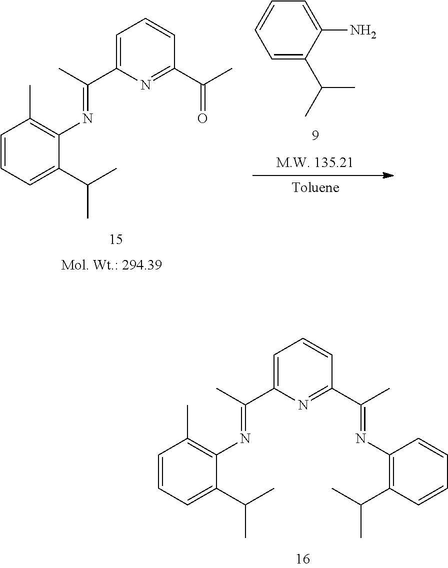

### (2-Isopropyl-6-methyl-phenyl)-(1-{6-[1-(2-isopropyl-phenylimino)-ethyl]-pyridin-2-yl}-ethylidene)-amine (16)

4.0 g (0.0135 mol) of 1-{6-[1-(2-isopropyl-6-methyl-phenylimino)-ethyl]-pyridin-2-yl}-ethanone 15, 2.76 g (0.0204 mol) of 2-Isopropyl-phenylamine 9 (4.0 g, 0.0135 mol), 100 g of fresh molecular sieves, and 100 ml of toluene were kept at 100° C. for 3 days under a flow of nitrogen. The solvent was removed in a rotary evaporator and the residue was recrystallized from 10 ml of ethanol. The yield of (2-Isopropyl-6-methyl-phenyl)-(1-{6-[1-(2-isopropyl-phenylimino)-ethyl]-pyridin-2-yl}-ethylidene)-amine 16 was 4.83 g (87%) as a yellow solid. ¹³C NMR (500 MHz, CD₂Cl₂, TMS (selected signals)): δ 166.9 (C═N), 166.2 (C═N).

### Example 6

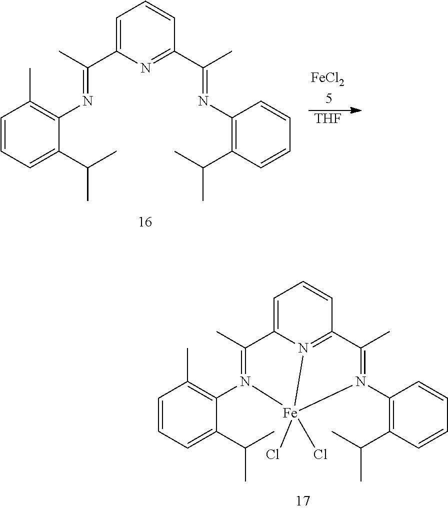

### (2-Isopropyl-6-methyl-phenyl)-(1-{6-[1-(2-isopropyl-phenylimino)-ethyl]-pyridin-2-yl}-ethylidene)-amine iron (II) chloride (17)

2.42 g (0.0059 mol) of (2-Isopropyl-6-methyl-phenyl)-(1-{6-[1-(2-isopropyl-phenylimino)-ethyl]-pyridin-2-yl}-ethylidene)-amine 16 (2.42 g, 0.0059 mol) was added in one portion to the suspension of 031 g (0.0056 mol) of iron (II) chloride in 40 ml of THF at ambient temperature under nitrogen glove box. The reaction mixture was stirred for 12 h. The resultant blue solid was filtered and washed by 50 ml of pentanes three times and dried under 1-mm vacuum overnight. The yield of (2-isopropyl-6-methyl-phenyl)-(1-{6-[1-(2-isopropyl-phenylimino)-ethyl]-pyridin-2-yl}-ethylidene)-amine iron (II) chloride 17 was 2.29 g (76%).

### Example 7

The iron complexes made in Examples 3 and 6 were used to oligomerize ethylene. The oligomerizations were run in a 1 l Autoclave Engineering Zipperclavet® recirculating batch reactor using 700 ml of o-xylene as the solvent. The iron complexes were activated using modified methylaluminoxane 3A, and ratios of the aluminoxane to Fe (Al/Fe) are given in Table 2. In all cases there was a very large excess of the aluminoxane. After 30-60 min the oligomerization was quenched by decreasing the ethylene pressure and cooling the reactor by passing cold water through the jacket. The Schulz-Flory constants were obtained in the standard manner by analyzing the process mixture by chromatography for α-olefins, measuring those olefins having 4 to about 30 carbon atoms, and using appropriate standards and corrections factors, calculating the amount of each olefin and then calculating the best fit Schulz-Flory constant. Temperatures at which the oligomerizations were carried out and the resulting Schulz-Flory constants are given in Table 2.

TABLE 2

| Iron | Temp, | | |
|---|---|---|---|
| Complex | ° C. | Al/Fe | SFC |
| 10 | 120 | 2,880 | 0.82 |
| | 100 | 7,190 | 0.80 |
| 17 | 85 | 24,640 | 0.85 |

1.-21. (canceled)

22. A process for the manufacture of a series of α-olefins of the formula R⁶⁰CH═CH₂, comprising, contacting ethylene, optionally one or more activators and/or cocatalysts, and an iron complex of a ligand of the formula: 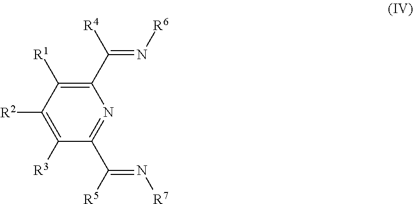
wherein:
R¹, R², and R³ are each independently hydrogen, hydrocarbyl, substituted hydrocarbyl, or an inert functional group, provided that any two of R¹, R², and R³ vicinal to one another taken together may form a ring;
R⁴ and R⁵ are each independently hydrogen, hydrocarbyl, substituted hydrocarbyl, or an inert functional group provided that R¹ and R⁴ and/or R³ and R⁵ taken together may form a ring;
R⁶⁰ is n-alkyl containing an even number of carbon atoms;
R⁶ is 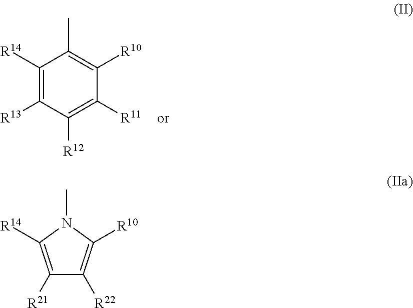
and R⁷ is 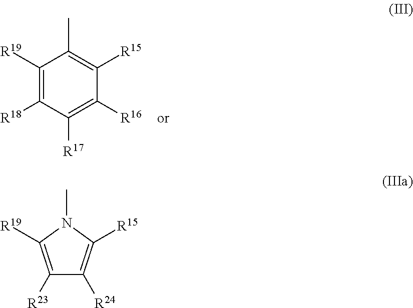
wherein:
R¹⁰, R¹⁴, and R¹⁵ are each independently hydrocarbyl, substituted hydrocarbyl, or a functional group other than fluoro, provided that at least one of R¹⁰, R¹⁴, and R¹⁵ is a secondary carbon group and/or a tertiary carbon group;
R¹¹ to R¹³, R¹⁶ to R¹⁸, and R²¹ to R²⁴ are each independently hydrogen hydrocarbyl, substituted hydrocarbyl, or a functional group, and
R¹⁹ is hydrogen or fluoro;
and further provided any two of R¹⁰ through R¹⁹ vicinal to one another may form a ring.

23. The process as recited in claim 22 wherein R¹⁰, and R¹⁵ are each independently alkyl containing 1 to 12 carbon atoms and R¹⁴ is hydrogen.

24. The process as recited in claim 22 wherein R⁴ and R⁵ are both methyl or both hydrogen.

25. The process as recited in claim 22 wherein at least two of R¹⁰, R¹⁴, and R¹⁵ are each independently a secondary carbon group and/or a tertiary carbon group.

26. The process as recited in claim 22 wherein all of R¹⁰, R¹⁴, and R¹⁵ are each independently a secondary carbon group and/or a tertiary carbon group.

27. The process as recited in claim 22 wherein said activator is present.

28. The process as recited in claim 27 wherein said activator is an alkylaluminum compound.

29. The process as recited in claim 22 wherein the process is carried out in the gas phase.

30. The process as recited in claim 22 wherein the process is carried out in the liquid phase.

31. The process as recited in claim 22 wherein the process is a solution process.

32. A compound of the formula 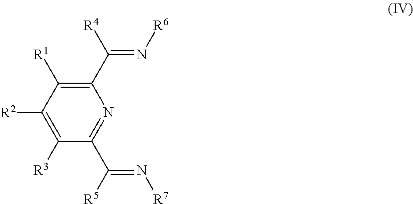
wherein:
R¹, R², and R³ are each independently hydrogen, hydrocarbyl, substituted hydrocarbyl, or an inert functional group, provided that any two of R¹, R², and R³ vicinal to one another, taken together, may form a ring;
R⁴ and R⁶ are each independently hydrogen, hydrocarbyl, substituted hydrocarbyl, or an inert functional group provided that R¹ and R⁴ and/or R³ and R⁵ taken together may form a ring;
R⁶⁰ is n-alkyl containing an even number of carbon atoms;
R⁶ is: 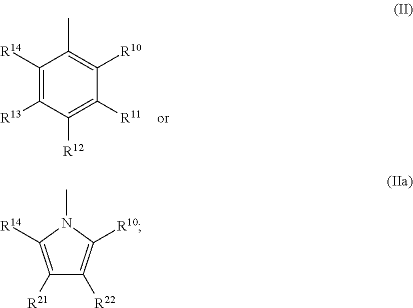
and R⁷ is: 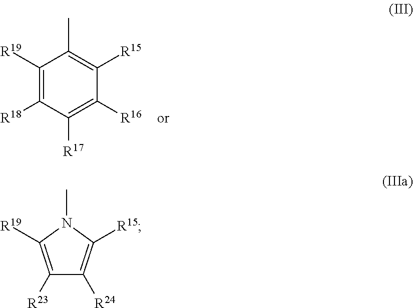
and wherein:
R¹⁰, R¹⁴, and R¹⁵ are each independently hydrocarbyl, substituted hydrocarbyl, or a functional group other than fluoro, provided that at least one of R¹⁰, R¹⁴, and R¹⁵ is a secondary carbon group and/or a tertiary carbon group;
R¹¹ to R¹³, R¹⁶ to R¹⁸, and R²¹ to R²⁴ are each independently hydrogen hydrocarbyl, substituted hydrocarbyl, or a functional group, and
R¹⁹ is hydrogen or fluoro;
and further provided
any two of R¹⁰ through R¹⁹ vicinal to one another may form a ring;
that R¹⁰, R¹⁴ and R¹⁵ are not all t-butyl;
and that R¹⁰ and R¹⁴ are not t-butyl when R¹⁵ is methyl.

33. The compound as recited in claim 32 wherein R¹⁰, R¹⁴, and R¹⁵ are each independently alkyl containing 1 to 12 carbon atoms and R¹⁴ is hydrogen.

34. The compound as recited in claim 32 wherein R⁴ and R⁵ are both methyl or both hydrogen.

35. The compound as recited in claim 32 wherein at least two of R¹⁰, R¹⁴, and R¹⁵ are each independently a secondary carbon group and/or a tertiary carbon group.

36. The compound as recited in claim 32 wherein all of R¹⁰, R¹⁴, and R¹⁵ are each independently a secondary carbon group and/or a tertiary carbon group.

37. The compound as recited in claim 32 wherein R⁶ is (II) and R⁷ is (III).

38. An iron complex of the compound of claim 32.

39. The iron complex of claim 38 wherein at least two of R¹⁰, R¹⁴, and R¹⁵ are each independently a secondary carbon group and/or a tertiary carbon group.

40. The iron complex od claim 38 wherein all of R¹⁰, R¹⁴, and R¹⁵ are each independently a secondary carbon group and/or a tertiary carbon group.

41. The iron complex as recited in claim 39 which contains one iron atom and one molecule of (IV).
